(19) United States
(12) Patent Application Publication
(10) Pub. No.: US 2008/0248518 A1
(43) Pub. Date: Oct. 9, 2008

(54) E.coli and salmonella test kit

(76) Inventor: Chandravadan Patel, Laguna Niguel, CA (US)

Correspondence Address: AVERILL & VARN, 8244 PAINTER AVE., WHITTIER, CA, 90602, US

(21) Appl. No.: 11/784,593

(22) Filed: Apr. 5, 2007

(51) Int. Cl. C12Q 1/04

(52) U.S. Cl. 435/34

(57) ABSTRACT

A method for testing for the presence of bacteria in the field includes using a mixer and frother or a reagent to disrupt bacteria cells. A sample is collected from a source suspected to be contaminated by bacteria. A mixer and frother or similar mixer is used to mix the sample, or a reagent is added to the sample, to form an elute by disrupting bacteria cells present in the sample to expose bacteria enzymes. A reagent pad is immersed in the elute and then withdrawn from the elute, or a drop of the elute is deposited on a reagent pad. After a waiting period, the reagent pad is observed to determine if the bacteria is present in or on the source. The source may be a body of water, produce, prepared food, a food preparation area, or food processing equipment. The reagent is generally a lysis reagent.

### BACKGROUND OF THE INVENTION

The present invention relates to tests for the presence of bacteria and in particular to a test kit for detecting the presence of Escherichia coli (E.coli) and salmonella bacteria on produce.

While many forms of bacteria are either harmless or beneficial to humans, some bacteria is potentially harmful or even fatal in sufficient quantities. Two of the most common harmful bacterium in the United States are E.coli and salmonella. Two types of samonella are common in the United States, salmonella typhimurium and salmonella enteritidis. Salmonella may be present in dairy, poultry and meat products, and Salmonella may grow on most food. Chickens and eggs are particularly high risk hosts for salmonella. Salmonella is often spread during food processing and thus be present on food not generally associated with salmonella, and contaminated foods usually look and smell normal. Food poisoning resulting from salmonella is called salmonellosis and is most common in young children, older adults, and individuals with impaired immune systems. Left untreated, salmonellosis may prove fatal.

There are various forms of E.coli, some helpful to humans, and some harmful. Along with other helpful bacteria, E.coli is present in the intestines of a healthy human and performs useful functions along with other bacterium, for example, producing vitamins we need to remain healthy. Unfortunately, one variety of E.coli, E.coli O157:H7, produces a harmful toxin. The toxin is a protein which causes severe damage to the cells that line the wall of the intestine and results in loss of water and salts, and because blood vessels are also damaged, hemorrhaging. E.coli is most commonly found in meat, but the E.coli from meat may contaminate other produce through various means.

Salmonella and E.coli may be detected using an enzyme detection test. The enzymes are present inside the bacteria cell, and known tests for salmonella and E.coli require mechanically breaking the cell open using a centrifuge or similar mechanical apparatus in a lab. There is often a desire to perform a quick field test and reduce costs associated with delays.

### BRIEF SUMMARY OF THE INVENTION

The present invention addresses the above and other needs by providing a method for testing for the presence of bacteria in the field, which method includes using a mixer and frother or a reagent to disrupt bacteria cells. A sample is collected from a source suspected to be contaminated by bacteria. A mixer and frother is used to mix the sample, or a reagent is added to the sample, to disrupt bacteria cells present in the sample to expose bacteria enzymes. A reagent pad is immersed in the sample and then withdrawn from the sample, or a drop of the sample is deposited on the reagent pad. After a waiting period, the reagent pad is observed to determine if the bacteria is present in or on the source. The source may be a body of water, produce, prepared food, a food preparation area, or food processing equipment. The reagent is generally a lysis reagent.

In accordance with one aspect of the invention, there is provided a method for testing for the presence of E.coli bacteria and/or for the presence of salmonella bacteria in the field. The method includes obtaining a test sample suspected to include bacteria contamination and adding a lysis reagent to the test sample to form an elute, thereby causing the release and/or exposure of enzymes from E.coli and/or salmonella bacteria present in the test sample. A first reagent pad is immersed into the elute and withdrawn to test for E.coli bacteria and/or a second reagent pad is immersed into the elute and then withdrawn to test for salmonella bacteria. A period of time is allowed for the reagent pad(s) to react to the presence of bacteria enzymes in the elute and then the reagent pads are observed for a change in color to determine if E.coli bacteria and/or salmonella bacteria is present. Generally, approximately six drops of lysis reagent is added to the test sample.

In another embodiment of the method of the present invention, there is provided a method for testing for the presence of E.coli bacteria and/or for the presence of salmonella bacteria in the field. The method includes obtaining a test sample suspected to include bacteria contamination and mixing the test sample using a mixer and frother to form an elute, thereby causing the release and/or exposure of enzymes from E.coli and/or salmonella bacteria present in the test sample. A first reagent pad is immersed into the elute and withdrawn to test for E.coli bacteria and/or a second reagent pad is immersed into the elute and then withdrawn to test for salmonella bacteria. A period of time is allowed for the reagent pad(s) to react to the presence of bacteria enzymes in the elute and then the reagent pads are observed for a change in color to determine if E.coli bacteria and/or salmonella bacteria is present. Optionally, a small amount, for example, approximately three drops, of a lysis reagent may be added to the test sample before mixing with the mixer frother to reduce the time required for the test and/or to provide a more distinct change in color of the reagent pad(s).

### BRIEF DESCRIPTION OF THE SEVERAL VIEWS OF THE DRAWING

The above and other aspects, features and advantages of the present invention will be more apparent from the following more particular description thereof, presented in conjunction with the following drawings wherein:

FIG. 1 is a test strip for testing for the presence of bacteria in a sample.

FIG. 2 is a test cassette for testing for the presence of two bacteria types in the sample.

FIG. 3A is a method according to the present invention for testing prepared food using a reagent to disrupt bacteria cell walls.

FIG. 3B is a method according to the present invention for testing prepared food using a mixer and frother to break bacteria cell walls.

FIG. 4A is a method according to the present invention for testing surfaces using a reagent to disrupt bacteria cell walls.

FIG. 4B is a method according to the present invention for testing surfaces using a mixer and frother to break bacteria cell walls.

FIG. 5A is a method according to the present invention for testing samples from a body of water using a reagent to disrupt bacteria cell walls.

FIG. 5B is a method according to the present invention for testing samples from a body of water using a mixer and frother to break bacteria cell walls.

FIG. 6A is a method according to the present invention for testing samples of material using a reagent to disrupt bacteria cell walls.

FIG. 6B is a method according to the present invention for testing samples of material using a mixer and frother to break bacteria cell walls.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

The present invention is a method for testing for the presence of bacteria, for example, E.coli and/or samonella bacteria, in a body of water, on produce, food preparation facilities, food preparation equipment, and material in general. Known methods require obtaining a test sample, transporting the sample to a lab, and using expensive mechanical equipment at the lab to break the bacteria cell to expose bacteria enzymes. Known tests are then used to detect the presence of the target bacteria enzymes. The present invention allows testing in the field versus a lab and provides results quickly and easily.

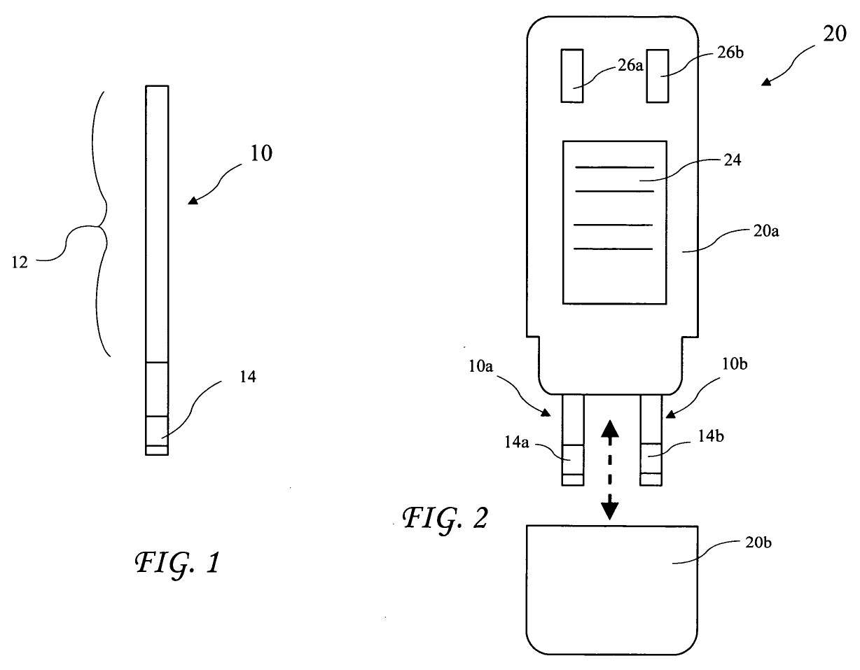

A test strip 10 for testing for the presence of bacteria in a sample is shown in FIG. 1. The test strip 10 is a narrow strip approximately 60 mm long and approximately four mm wide. A handle or upper portion 12 preferably includes an indication of the type of test or target (for example, targeting E.coli or samonella bacteria) the test strip 10 is directed to. A reagent pad 14 resides on a lower portion of the test strip 10. The reagent pad 14 is directed to detecting the presence of the test target, any change in color (for example towards blue) of the reagent pad 14 indicates that the target bacteria is present.

A typical test strip 10 may be constructed as follows. A reagent paper sheet (typically approximately 16 cm by approximately 16 cm) is obtained, for example from Schleicher & Schuell in Keene, N.H., or Whatman Company in England. The reagent paper is coated with a suitable regent mixture. A double sided tape, for example, 3M #415 from 3M Company in St. Paul, Minn., is attached on one side of the reagent paper sheet. The paper and tape combination is cut into approximately 4 mm wide by approximately 160 mm ribbons using a cutter to form a reagent paper ribbon from which the reagents pads 14 are formed.

A PVC plastic roll of approximately 1 mm thick PVC and approximately 60 mm width is used to provide the body of the test strips which the reagent pads 14 are attached to. The PVC is cut into approximately 300 mm by approximately 60 mm sheets. Paper covering an exposed side the double sided tape is removed from the reagent paper ribbon and the reagent paper ribbon is then attached to the surface of the 300 mm PVC sheet along one of the 300 mm edges. The PVC test strip with the reagent paper ribbon attached is then cut into approximately 4 mm wide strips using a cutter. The final size of the test strip 10 with reagent pad 14 is thus approximately 4 mm by approximately 60 mm. A similar process is carried out for both reagent strips (E.coli & Salmonella) except the active reagents on the test strips will be different.

The suitable reagent mixture used to identify the presence of bacteria is an enzyme substrate or a combination of enzyme substrates. Enzymes released from bacteria cells act upon the enzyme substrate to cause a change in the substrate and producing a color change. Examples of suitable enzyme substrates for detecting E.coli bacteria are:

4-nitrophenyl-alpha-d-galactopyranoside;

4-nitrophenyl-beta-d-glucuronic acid; phenolphthalein-beta-d-glucuronic acid;

phenolphthalein-beta-d-glucuronic acid, sodium salt;

phenolphthalein monophosphate, bis(cyclohexylammonium) salt;

5-bromo-4-chloro-3-indoxyl-beta-d-glucuronic acid, cyclohexylammonium salt;

5-bromo-6-chloro-3-indoxyl-beta-d-glucuronic acid, cyclohexylammonium salt;

5-bromo-4-chloro-3-indoxyl-beta-d-glucuronic acid, sodium salt anhydrous;

5-bromo-4-chloro-3-indoxyl-beta-d-glucuronic acid, sodium salt;

5-bromo-3-indoxyl-beta-d-galactopyranoside;

5-bromo-3-indoxyl-beta-d-glucuronic acid, cyclohexylammonium salt;

6-bromo-2-naphthyl-alpha-d-galactopyranoside;

6-bromo-2-naphthyl-beta-d-galactopyranoside;

6-bromo-2-naphthyl-beta-d-glucuronic acid;

6-chloro-3-indoxyl-beta-d-glucuronic acid, cyclohexylammonium salt;

2-chloro-4-nitrophenyl-beta-d-galactopyranoside;

8-hydroxyquinoline-beta-d-galactopyranoside; and

8-hydroxyquinoline-beta-d-glucuronic acid, sodium salt.

Examples of suitable enzyme substrates for detecting samonella bacteria are:

5-Bromo-4-chloro-3-indoxyl-3-acetate;

5-Bromo-6-chloro-3-indoxyl-3-acetate;

5-Bromo-4-chloro-3-indoxyl butyrate

5-Bromo-6-chloro-3-indoxyl butyrate

5-Bromo-6-chloro-3-indoxyl caprate;

5-Bromo-4-chloro-3-indoxyl caprylate;

5-Bromo-6-chloro-3-indoxyl caprylate;

5-Bromo-4-chloro-3-indoxyl palmitate; and

5-Bromo-3-indoxyl-3-acetate.

Two or more test strips 10a and 10b may be housed in a test cassette 20 as shown in FIG. 2 for easy use. The specific test strip type (indicator of the target bacteria) is visible through windows 26a & 26b of cassette 20. The bottoms of each test strip 10a and 10b extend below a cassette main body 20a exposing the reagent pads 14a and 14b when a cassette cap 20b is removed as shown in FIG. 2. The reagent pads 14a and 14b may then be immersed in a sample, or drops of the sample may be deposited on the reagent pads 14a and 14b, to perform a test for bacteria. When not in use, the cap 20b may be placed over the exposed ends of the test strips 10a and 10b to prevent damage or contamination of the reagent pads 14a and 14b.

Unfortunately, known methods for testing for the presence of harmful bacteria require transporting a sample to a lab for processing and do not allow a fast and easy test to be timely performed in the field (i.e., location where the sample is collected). The methods of the present invention introduce the use of either a mixer and frother (or any similar hand mixer, counter top mixer, and the like) and/or a lysis reagent for breaking the bacteria cells open to release or expose the bacteria enzymes and allows simple and timely testing in the field, versus requiring transporting the test sample to a lab. Previous methods relied on the use of lab equipment unsuitable for use in the field, but the present invention unexpectedly recognized that a lysis reagent alone, a lysis reagent and a simple and inexpensive mixer and frother or other similar mixer, or a mixer and frother or other similar mixer alone may be used in the field to expose bacteria enzymes for testing. Use of the lysis reagent alone produces good results, but is somewhat costly. The combined use of a smaller quantity of the lysis reagent combined with mixing with the mixer and frother reduces cost and provides good results. Use of the mixer and frother alone provides satisfactory results but may take longer.

The lysis reagent disrupts the bacteria cell wall to release or expose the bacteria enzymes for testing. An example of a suitable lysis reagent is Fastbreak™ lysis reagent made by Promega in Madison, Wis. When a lysis reagent is used alone to release or expose the bacteria enzymes, approximately six drops of the lysis reagent is used with an approximately two to approximately three ounce test sample. While a lysis reagent may be used alone to expose bacterial enzymes, a smaller amount of the lysis reagent may be used when the resulting elute is mixed using, for example, the mixer and frother or similar mixer. When the resulting elute is mechanically mixed, the amount of lysis reagent may be reduced by approximately 50% to, for example, approximately three drops of the lysis reagent is used with an approximately two to approximately three ounce test sample, thereby achieving a substantial cost savings.

A mixer and frother is particularly suitable for field use because of small size and low cost. Such mixer frother is easily carried to the filed and easily replaced if damaged, lost, or stolen. A suitable mixer and frother is the Mini Mixer mixer and frother made by Norpro in Everett, Wash. The mixer and frother preferably breaks the cell walls of both E.coli and samonella bacteria without damaging the enzymes. Other small mixers suitable for use in the field may readily be substituted for the Mini Mixer, and methods similar to the present invention merely substituting a comparable mixer are intended to come within the scope of the present invention.

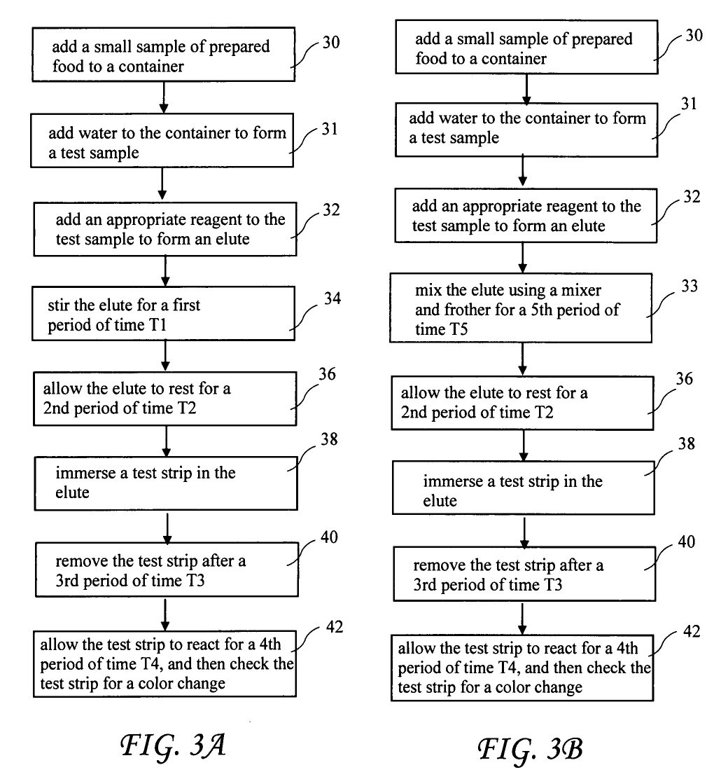

A method according to the present invention for testing prepared food using a reagent to disrupt the bacterial cell wall is described in FIG. 3A. Add a small sample of a prepared food to a container at step 30. Add water to the container to form a test sample at step 31. Add a lysis reagent to the test sample to form an elute at step 32. Stir the elute (or otherwise mix) for a first period of time T1 at step 34. The first period of time T1 is preferably at least approximately 30 seconds. Allow the elute to rest for a second period of time T2 at step 36. The second period of time T2 is preferably approximately 20 seconds to approximately 30 seconds. Associate a test strip 10, or reagent pads 14a and 14b extending from the cartridge 20, with the elute by immersing into the elute at step 38. Remove the test strip 10, or reagent pads 14a and 14b, from the elute after a third period of time T3, at step 40. The third period of time T3 is preferably between approximately 60 seconds and approximately 90 seconds. Alternatively, a drop of the elute may be deposited on the reagent pad(s) 14, 14a, or 14b. Observe the reagent pad(s) 14, 14a, and/or 14b for a color change after a fourth period of time T4 at step 42. The fourth period of time T4 is preferably between approximately 20 minutes and approximately 45 minutes for detecting salmonella, and between approximately 30 minutes and approximately 60 minutes for detecting E.coli. If the reagent pad changes color or begins to change color, the presence of bacteria in the test sample has been detected.

The method described in FIG. 3A does not include using a mixer and frother or similar mixer to help break open bacterial cells to expose the bacteria enzymes for detection of the presence of the bacteria. Because a mixer and frother or similar mixer is not used, a greater amount of reagent is generally required at step 32, and preferably, approximately six drops of a lysis reagent are added to the test sample at step 32.

A method according to the present invention for testing prepared food using a mixer and frother to open the bacterial cell wall is described in FIG. 3B. Add a small sample of a prepared food to a container at step 30. Add water to the container to result in a total test sample size of preferably between approximately five ml and approximately ten ml at step 31. Optionally, add an appropriate reagent to the test sample to form an elute at step 32. Mix the elute using the mixer and frother or similar mixer for a fifth period of time T5 at step 33. The fifth period of time T5 is preferably at least approximately five minutes and is more preferably between approximately five minutes and approximately ten minutes. Allow the mixed elute to rest for the second period of time T2 at step 36. Associate a test strip 10, or reagent pads 14a and 14b extending from the cartridge 20, with the elute by immersing into the elute at step 38. Remove the test strip 10, or reagent pads 14a and 14b from the elute after the third period of time T3, at step 40. The third period of time T3 is preferably between approximately 60 seconds and approximately 90 seconds. Alternatively, the test strip 10, or reagent pads 14a and 14b, may be associated with the elute by depositing a drop of the elute on the reagent pad(s) 14, 14a, and/or 14b. Observe the reagent pad(s) 14, 14a, and/or 14b for as much as the fourth period of time T4 for a color change at step 42. The fourth period of time T4 is preferably between approximately 20 minutes and approximately 45 minutes for detecting salmonella, and between approximately 30 minutes and approximately 60 minutes for detecting E.coli. If the reagent pad changes color or begins to change color, the presence of bacteria has been detected.

Step 32 is optional in the method of FIG. 3B and the method may be performed without step 32. Adding the reagent at step 32 generally reduces the time required for the test and in the instance where the elute is mixed with the mixer and frother or similar mixer at step 33, and a smaller amount of reagent is generally required than when the mixer and frother or similar mixer is not used. Generally, approximately three drops of an appropriate reagent are added to the test sample at step 32 when the elute is subsequently mixed with the mixer and frother or similar mixer at step 33. In the instance where the lysis reagent is not added, step 33 comprises mixing the test sample with the mixer and frother. In either instance, the result of adding the lysis reagent, or the result of mixing the test sample with the mixer and frother, is herein referred to as an “elute.”

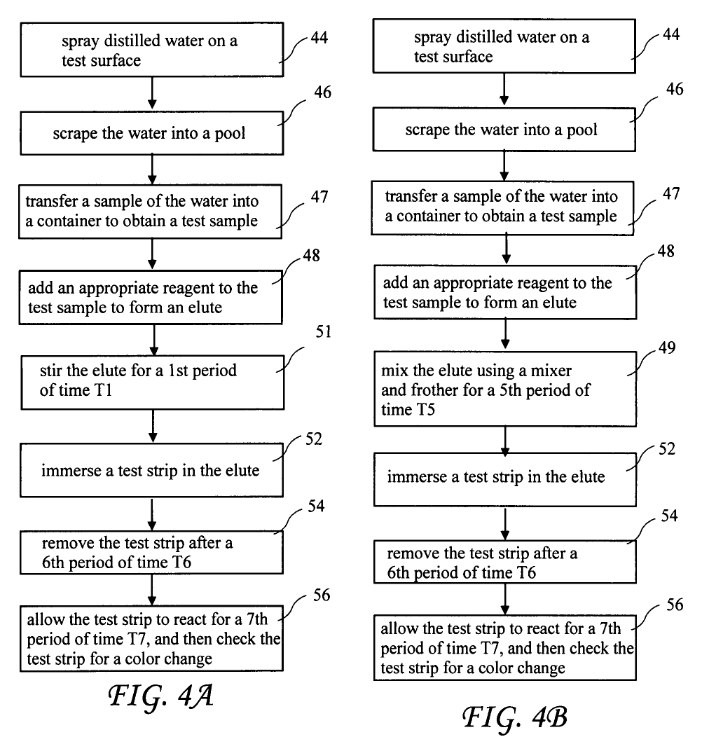

A method according to the present invention for testing food preparation areas, food preparation tools, food preparation equipment, and the like using a reagent to disrupt the bacterial cell wall, is described in FIG. 4A. Spray distilled water (or an equivalent) onto a surface at step 44. Scrape or gather the water and any material mixed with the water into a pool to form a sample at step 46. Transfer the sample into a container to obtain a test sample at step 47. Add a lysis reagent to the test sample to form an elute at step 48. Stir (or otherwise mix) the elute for the first period of time T1 at step 51. The first period of time T1 is preferably at least approximately 30 seconds. Associate a test strip, 10 or reagent pads 14a and 14b extending from the cartridge 20, with the elute by immersing into the elute at step 52. Remove the test strip 10 or reagent pads 14a and 14b, from the elute and place on a non-absorbent clear surface after a sixth period of time T6, at step 54. The sixth period of time T6 is preferably at least approximately 30 seconds. Alternatively, the test strip 10, or reagent pads 14a and 14b, may be associated with the elute by depositing a drop of the elute on the reagent pad(s) 14, 14a, and/or 14b. Observe the reagent pad(s) 14, 14a, and/or 14b for as much as a seventh period of time T7 for a color change at step 56. The seventh period of time T7 is preferably between approximately 20 minutes and approximately 45 minutes for detecting salmonella, and between approximately 30 minutes and approximately 60 minutes for detecting E.coli. If the reagent pad changes color or begins to change color, the presence of bacteria in the test sample has been detected.

The method described in FIG. 4A does not include using a mixer and frother or similar mixer to help break open bacterial cells to expose the bacteria enzymes for detection of the presence of the bacteria. Because a mixer and frother or similar mixer is not used, a greater amount of reagent is generally required at step 48, and preferably, approximately six drops of a lysis reagent are added to the container at step 48.

A method according to the present invention for testing food preparation areas, food preparation tools, food preparation equipment, and the like using a mixer and frother to break the bacterial cell wall, is described in FIG. 4B. Spray distilled water (or an equivalent) onto a surface at step 44. Scrape or gather the water, and any material mixed with the water, into a pool to form a sample at step 46. Transfer the sample into a container to obtain a test sample at step 47. Optionally, add a lysis reagent to the test sample to form an elute at step 48. Mix the elute using the mixer and frother or similar mixer for a fifth period of time T5 at step 49. The fifth period of time T5 is preferably at least approximately five minutes and is more preferably between approximately five minutes and approximately ten minutes. Associate a test strip 10, or reagent pads 14a and 14b extending from the cartridge 20, with the elute by immersing into the elute at step 52. Remove the test strip 10, or reagent pads 14a and 14b, from the elute and place on a non-absorbent clear surface after a sixth period of time T6, at step 54. The sixth period of time T6 is preferably at least approximately 30 seconds. Alternatively, the test strip 10, or reagent pads 14a and 14b, may be associated with the elute by depositing a drop of the elute on the reagent pad(s) 14, 14a, and/or 14b. Observe the reagent pad(s) 14, 14a, and/or 14b for a color change after a seventh period of time T7 at step 56. The seventh period of time T7 is preferably between approximately 20 minutes and approximately 45 minutes for detecting salmonella, and between approximately 30 minutes and approximately 60 minutes for detecting E.coli. If the reagent pad changes color or begins to change color, the presence of bacteria in the test sample has been detected.

Step 48 is optional in the method of FIG. 4B and the method may be performed without step 48. Adding the reagent at step 48 generally reduces the time required for the test and in the instance where the elute is mixed with the mixer and frother or similar mixer at step 49, and a smaller amount of reagent is generally required than when the mixer and frother or similar mixer is not used. Generally, approximately three drops of an appropriate reagent are added to the container at step 48 when the elute is subsequently mixed with the mixer and frother or similar mixer at step 49. In the instance where the lysis reagent is not added, step 49 comprises mixing the test sample with the mixer and frother to form an elute. In either instance, the result of adding the lysis reagent, or the result of mixing the test sample with the mixer and frother, is referred to as an “elute.”

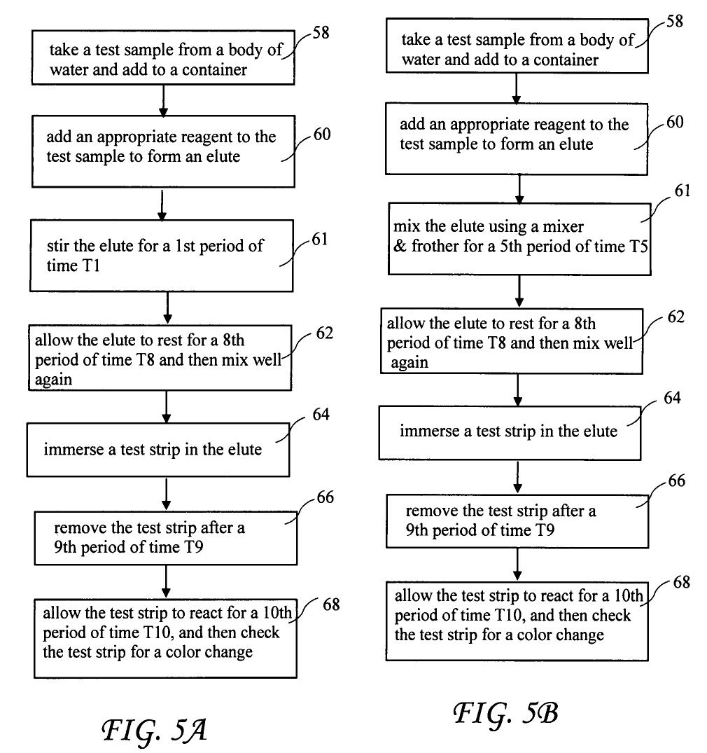

A method according to the present invention for testing samples from a body of water, using the reagent to disrupt the bacterial cell wall, is described in FIG. 5A. Take a test sample from the body of water and transfer the sample into a container at step 58. Add a lysis reagent to the test sample to form an elute at step 60. Stir (or otherwise mix) the elute for the first period of time T1 at step 61. The first period of time T1 is preferably at least approximately 30 seconds. Allow the elute to rest for an eighth period of time T8 and then mix again at step 62. The eighth period of time T8 is preferably approximately ten minutes. Associate a test strip 10 or reagent pads 14a and 14b extending from the cartridge 20, with the elute by immersing the test strip 10, or reagent pads 14a and 14b, in the elute at step 64. Remove the test strip 10, or reagent pads 14a and 14b extending from the cartridge 20, from the elute after a ninth period of time T9 at step 66. The ninth period of time T9 is preferably at least approximately 30 seconds Alternatively, a drop of the elute may be deposited on the reagent pad(s) 14. Observe the reagent pad(s) 14, 14a, and/or 14b for a color change after a tenth period of time T10 at step 68. The tenth period of time T10 is preferably between approximately 20 minutes and approximately 45 minutes for detecting salmonella, and between approximately 30 minutes and approximately 60 minutes for detecting E.coli. If the reagent pad changes color or begins to change color, the presence of bacteria in the test sample has been detected.

The method described in FIG. 5A does not include using a mixer and frother or similar mixer to help break open bacterial cells to expose the bacteria enzymes for detection of the presence of the bacteria. Because a mixer and frother or similar mixer is not used, a greater amount of reagent is generally required at step 60, and preferably, approximately six drops of a lysis reagent are added to the container at step 60.

A method according to the present invention for testing samples from a body of water, using the mixer and frother to break the bacterial cell wall, is described in FIG. 5B. Take a test sample from the body of water and transfer the sample into a container at step 58. Optionally, add an appropriate reagent to the test sample to form an elute at step 60. Mix the elute well using the mixer and frother for the fifth period of time T5 at step 61. The fifth period of time T5 is preferably at least approximately five minutes and is more preferably between approximately five minutes and approximately ten minutes. Allow the elute to rest for an eighth period of time T8 and then well mix again at step 62. The eighth period of time T8 is preferably approximately ten minutes. Associate a test strip 10, or reagent pads 14a and 14b extending from the cartridge 20, by immersing into the elute at step 64. Remove the test strip 10, or reagent pads 14a and 14b extending from the cartridge 20, from the elute after the ninth period of time T9 at step 66. Alternatively, a drop of the elute may be deposited on the reagent pad(s) 14, 14a, and/or 14b. Observe the reagent pad(s) 14, 14a, and/or 14b for a color change after a tenth period of time T10. The tenth period of time T10 is preferably between approximately 20 minutes and approximately 45 minutes for detecting salmonella, and between approximately 30 minutes and approximately 60 minutes for detecting E.coli. If the reagent pad changes color or begins to change color, the presence of bacteria in the test sample has been detected.

Step 60 is optional in the method of FIG. 5B and the method may be performed without step 60. Adding a lysis reagent at step 60 generally reduces the time required for the test and in the instance where the mixture is mixed with the mixer and frother or similar mixer at step 61, and a smaller amount of the lysis reagent is generally required than when the mixer and frother or similar mixer is not used. Generally, approximately three drops of a lysis reagent is added to the container at step 60 when the mixture is subsequently mixed with the mixer and frother or similar mixer at step 61. In the instance where the lysis reagent is not added, step 61 comprises mixing the test sample with the mixer and frother. In either instance, the result of adding the lysis reagent, or the result of mixing the test sample with the mixer and frother, is referred to as an “elute.”

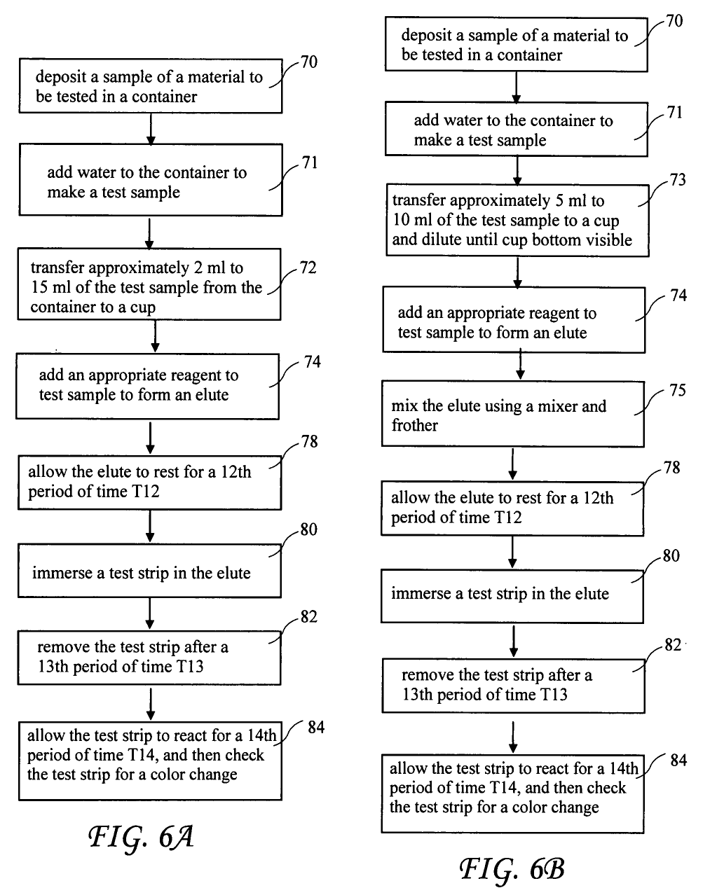

A method according to the present invention for testing material such as lettuce, cabbage, cow manure, pig manure, chicken manure, donkey manure, and the like using a reagent to disrupt the bacterial cell wall is described in FIG. 6A. Add between approximately 3 ounces and approximately 5 ounces of the material to a container at step 70. Add between approximately 50 ml and 200 ml of distilled water to the container to form a test sample at step 71. Transfer between approximately 2 ml and 15 ml of the test sample to a cup at step 72. Add approximately six drops of lysis reagent to the test sample to form an elute at step 74. Allow the elute to rest for a twelfth period of time T12 at step 78. The twelfth period of time T12 is preferably approximately five minutes. Associate a test strip 10, or reagent pads 14a and 14b extending from the cartridge 20, with the elute by immersing in the elute at step 80. Remove the test strip 10, or reagent pads 14a and 14b, from the elute after a thirteenth period of time T13, at step 82. The thirteenth period of time T13 is preferably between approximately 60 seconds and approximately 75 seconds. Alternatively, two drops of the elute may be deposited on the reagent pad(s) 14, 14a, or 14b. Observe the reagent pad(s) 14, 14a, and/or 14b after a fourteenth period of time T14 for a color change at step 84. The fourteenth period of time T14 is preferably between approximately 20 minutes and approximately 45 minutes for detecting salmonella, and between approximately 30 minutes and approximately 60 minutes for detecting E.coli. If the reagent pad changes color or begins to change color, the presence of bacteria has been detected.

The method described in FIG. 6A does not include using a mixer and frother or similar mixer to help break open bacterial cells to expose the bacteria enzymes for detection of the presence of the bacteria. Because a mixer and frother or similar mixer is not used, a greater amount of reagent is generally required at step 74 than required when a mixer and frother is used, and preferably, approximately six drops of a lysis reagent are added to the container at step 74.

A method according to the present invention for testing material such as lettuce, cabbage, cow manure, pig manure, chicken manure, donkey manure, and the like using a mixer frother to disrupt the bacterial cell wall is described in FIG. 6B. Add between approximately 3 ounces and approximately 5 ounces of the material to a container at step 70. Add between approximately 50 ml and 200 ml of distilled water to the container to form a sample at step 71. Transfer between approximately 5 ml and 10 ml of the sample to a cup and dilute the sample until the bottom of the cup is visible to form a test sample at step 73. Optionally, add a lysis reagent to the test sample to form an elute at step 74. Mix the elute with a mixer frother for between approximately three seconds and approximately five seconds to form an elute at step 75. Allow the elute to rest for a twelfth period of time T12 at step 78. The twelfth period of time T12 is preferably approximately five minutes. Associate a test strip 10, or reagent pads 14a and 14b extending from the cartridge 20, with the elute by immersing in the elute at step 80. Remove the test strip 10, or reagent pads 14a and 14b, from the elute after a thirteenth period of time T13, at step 82. The thirteenth period of time T13 is preferably between approximately 60 seconds and approximately 75 seconds. Alternatively, two drops of the elute may be deposited on the reagent pad(s) 14, 14a, or 14b. Observe the reagent pad(s) 14, 14a , and/or 14b after a fourteenth period of time T14 for a color change at step 84. The fourteenth period of time T14 is preferably between approximately 40 minutes and approximately 100 minutes for detecting salmonella, and between approximately 40 minutes and approximately 100 minutes for detecting E.coli. If the reagent pad changes color, the presence of bacteria has been detected.

Step 74 is optional in the method of FIG. 6B and the method may be performed without step 74. Adding the lysis reagent at step 74 generally reduces the time required for the test and in the instance where the mixture is mixed with the mixer and frother or similar mixer at step 75, and a smaller amount of reagent is generally required than when the mixer and frother or similar mixer is not used. Generally, approximately three drops of an appropriate reagent are added to the container at step 74 when the mixture is subsequently mixed with the mixer and frother or similar mixer at step 75. In the instance where the lysis reagent is not added, step 75 comprises mixing the test sample with the mixer and frother. In either instance, the result of adding the lysis reagent, or the result of mixing the test sample with the mixer and frother, is referred to as an “elute.”

In the methods described in FIGS. 3A, 3B, 4A, 4B, 5A, 5B, 6A and 6B, the mixer and frother is used to mix, and/or a lysis reagent is added, to release or expose enzymes from bacteria cells to allow testing for the enzymes. Methods with and without mixing benefit from adding a reagent to the test sample, however, less reagent is required when the test sample is mechanically mixed. Typically, six drops of reagent are sufficient without mechanical mixing, and three drops of reagent are sufficient to shorten the time required for the test when mechanical mixing is used. Further, although mechanical mixing using a mixer and frother or similar mixer was described above, any suitable mixer, hand mixer, counter top mixer, and the like is suitable for mechanically mixing the sample to expose bacterial enzymes for testing, and a method following steps in FIGS. 3B, 4B, 5B, and 6B using any similar mixer is intended to come within the scope of the present invention. An advantage of mixing using the mixer and frother, or equivalent mixer, is the portability and low cost of the mixer and frother. Such portability is a substantial advantage for use in the field versus a lab.

While the invention herein disclosed has been described by means of specific embodiments and applications thereof, numerous modifications and variations could be made thereto by those skilled in the art without departing from the scope of the invention set forth in the claims.

I claim:

1. A method for testing for the presence of bacteria, the method comprising:
obtaining a test sample suspected to include bacteria contamination;
processing the test sample in the field to release enzymes from the bacteria;
associating the processed sample with a reagent pad on a test strip;
allowing a period of time for the reagent pad to react to the presence of bacteria in the mixed mixture; and
observing for a change in the reagent pad to determine if the bacteria is present.

2. The method of claim 1, wherein processing the sample in the field to release enzymes from the bacteria comprises mixing the test sample using a hand held mixer for a fifth period of time T5.

3. The method of claim 2, wherein mixing the test sample using a hand held mixer for a fifth period of time T5 comprises mixing the test sample using a mixer and frother for a fifth period of time T5.

4. The method of claim 3, wherein mixing the test sample using a mixer and frother for a fifth period of time T5 comprises mixing the sample with a mixer and frother for the fifth period of time T5 between approximately five minutes and approximately ten minutes.

5. The method of claim 4, further including allowing the mixture to rest for a second period of time T2 after mixing the test sample using a mixer and frother for the period of time T5 and before associating the processed sample with the reagent pad.

6. The method of claim 5, wherein allowing the mixture to rest for a first period of time T2 comprises allowing the mixture to rest for the period of time T2 between approximately 20 seconds and approximately 30 seconds.

7. The method of claim 1, wherein processing the test sample in the field to release enzymes from the bacteria comprises adding approximately three drops of a reagent to the test sample to form an elute and then mixing the elute using a hand held mixer.

8. The method of claim 7, wherein processing the sample in the field to release enzymes from the bacteria comprises adding approximately three drops of a lysis reagent to the test sample to form an elute and then mixing the elute using a mixer and frother.

9. The method of claim 1, wherein processing the sample in the field to release enzymes from the bacteria comprises:
mixing a lysis reagent with the test sample to form an elute; and then allowing the elute to rest for a first period of time T2.

10. The method of claim 9, wherein allowing the elute to rest for the second period of time T2 comprises allowing the elute to rest for the period of time T2 of between approximately 20 seconds to approximately 30 seconds.

11. The method of claim 9, wherein mixing the lysis reagent with the test sample to form the elute comprises mixing approximately six drops of the lysis reagent with the test sample to form the elute.

12. The method of claim 1, wherein obtaining a test sample suspected to include bacteria contamination comprises:
adding a sample of prepared food to a container; and
adding water to the container.

13. The method of claim 1, wherein obtaining a test sample comprises:
applying a liquid to a surface in a food preparation area; and
collecting a portion of the liquid.

14. The method of claim 1, wherein obtaining a test sample comprises collecting a sample from a body of water.

15. The method of claim 1, wherein obtaining a test sample comprises depositing a sample of a material in a container.

16. The method of claim 15, wherein obtaining a test sample comprises depositing a sample of a material selected from the group consisting of lettuce, cabbage, cow manure, pig manure, chicken manure, and donkey manure, in a container and adding water.

17. A method for testing for the presence of bacteria, the method comprising:
obtaining a test sample suspected to include bacteria contamination;
mixing the test sample in the field using a mixer and frother for a fifth period of time T5 of at least approximately five minutes to form an elute, thereby causing the exposure of enzymes from the bacteria;
associating the elute with a reagent pad on a test strip;
allowing a period of time for the reagent pad to react to the presence of bacteria in the elute; and
observing for a change in the reagent pad to determine if the bacteria is present in the test sample.

18. The method of claim 17, further including the step of adding approximately three drops of a lysis reagent to the test sample before mixing with the mixer and frother.

19. A method for testing for the presence of bacteria, the method comprising:
obtaining a test sample suspected to include bacteria contamination;
mixing a lysis reagent with the test sample in the field to form an elute, thereby causing the exposure of enzymes from the bacteria;
associating the elute with a reagent pad;
allowing a period of time for the reagent pad to react to the presence of bacteria in the elute; and
observing for a change in the reagent pad to determine if the bacteria is present.

20. The method of claim 19, wherein mixing a lysis reagent with the test sample comprises mixing approximately six drops of the lysis reagent with the test sample.
